(19) United States
(12) Patent Application Publication
(10) Pub. No.: US 2023/0197263 A1
(43) Pub. Date: Jun. 22, 2023

(54) ENHANCED INFORMATION DURING PATIENT MONITORING

(71) Applicant: Welch Allyn, Inc., Skaneateles Falls, NY (US)

(72) Inventors: Jennifer Bergstrom, Portland, OR (US); Stacie L. Brough, Syracuse, NY (US); Christopher Long, Skaneateles Falls, NY (US); Tiffany Moon, Skaneateles Falls, NY (US); Thomas A. Myers, Syracuse, NY (US); Marcia Wawro, Skaneateles Falls, NY (US); Ching Yue Yeung, Skaneateles Falls, NY (US)

(21) Appl. No.: 18/067,303

(22) Filed: Dec. 16, 2022

Related U.S. Application Data
(60) Provisional application No. 63/265,787, filed on Dec. 21, 2021.

(51) Int. Cl. G16H 40/63

(52) U.S. Cl. CPC G16H 40/63

(57) ABSTRACT

A monitoring device receives an identifier for a caregiver, determines an access level for the monitoring device based upon the identifier of the caregiver, and generates a dynamic interface. The dynamic interface being configured based upon the access level of the caregiver. The monitoring devices accesses historical physiological information based upon the access level and suggests one or more alarm limits based upon the historical physiological information.

### BACKGROUND

Caregivers, such as nurses and physicians, use various types of healthcare equipment to assist with the task of providing healthcare to a patient. Some healthcare equipment can be designed to monitor various physiological parameters of a patient, such as blood pressure, pulse rate, and temperature. This healthcare equipment can be complicated to use and may not always provide all the information needed by the caregiver.

### SUMMARY

In general terms, the present disclosure relates to role-based access and patient data pull back. Various aspects are described in this disclosure, which include, but are not limited to, the following aspects.

One aspect relates to monitoring device, comprising: at least one processor; and at least one system memory encoding instructions which, when executed by the at least one processor, cause the monitoring device to: receive an identifier for a caregiver; determine an access level for the monitoring device based upon the identifier of the caregiver; generate a dynamic interface, the dynamic interface being configured based upon the access level of the caregiver; access historical physiological information based upon the access level; and suggest one or more alarm limits based upon the historical physiological information.

Another aspect relates to monitoring device, comprising: at least one processor; and at least one system memory encoding instructions which, when executed by the at least one processor, cause the monitoring device to: request historical physiological information from a second device; receive the historical physiological information; display the historical physiological information on the monitoring device; and suggest one or more alarm limits based upon the historical physiological information.

Another aspect relates to method for monitoring a patient, the method comprising: receiving an identifier for a caregiver; determining an access level for a monitoring device based upon the identifier of the caregiver; receiving historical physiological information for the patient; generating a dynamic interface, the dynamic interface being configured based upon the access level of the caregiver; displaying the historical physiological information, wherein display of the historical physiological information is limited by the access level of the caregiver; and suggesting one or more alarm limits based upon the historical physiological information.

### DESCRIPTION OF THE FIGURES

The following drawing figures, which form a part of this application, are illustrative of described technology and are not meant to limit the scope of the disclosure in any manner.

FIG. 1 shows an example of a system for monitoring a patient.

FIG. 2 shows an example of logical components of a monitoring device of the system of FIG. 1.

FIG. 3A shows an example of a user interface of the monitoring device of FIG. 2.

FIG. 3B shows another example of a user interface of the monitoring device of FIG. 2.

FIG. 4 shows an example of a method implemented by the monitoring device of FIG. 2.

FIG. 5 shows an additional example of logical components of the monitoring device of the system of FIG. 1.

FIG. 6 shows an example of a method implemented by the monitoring device of FIG. 5.

FIG. 7 shows an example of components of the monitoring device of FIG. 2.

### DETAILED DESCRIPTION

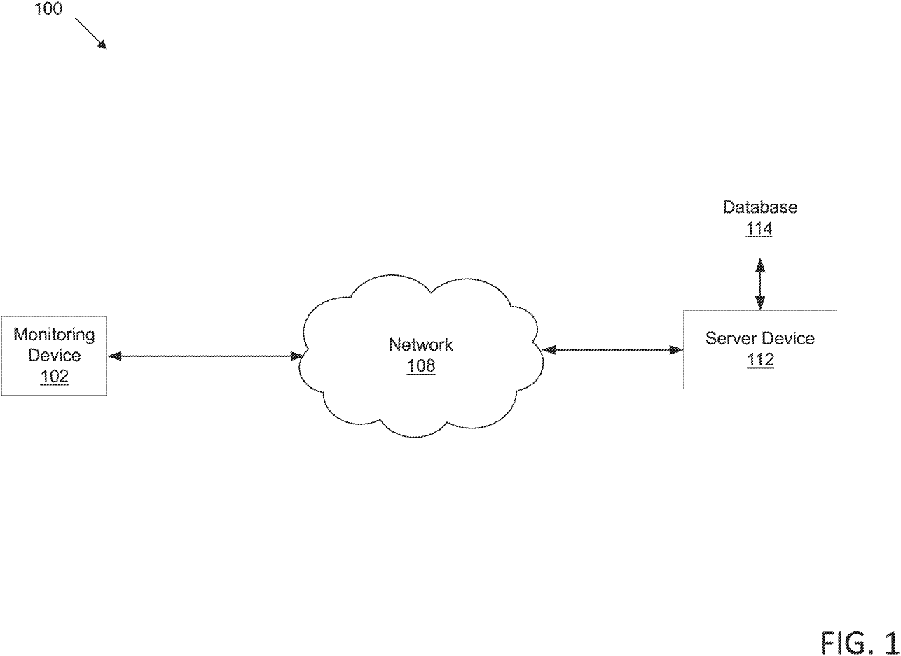

FIG. 1 is a schematic block diagram illustrating an example system 100 for enhancing the information that is provided during monitoring of a patient. In some embodiments, the information is tailored to the caregiver based upon the caregiver’s role. In other embodiments, historical physiological information is retrieved to further enhance the information that is displayed. Other combinations and configurations are possible.

The example system 100 includes a monitoring device 102 and a server device 112 connected to the monitoring device 102 through a network 108.

The monitoring device 102 functions to monitor various physiological parameters associated with the patient. More specifically, the monitoring device 102 includes multiple healthcare equipment (HCE) modules. Each of the HCE modules is configured to measure one or more physiological parameters of the patient.

Examples of these HCE modules include a temperature measurement module, an SpO2 module, and a non-invasive blood pressure (NIBP) module. The temperature measurement module can include a temperature probe designed to sense a body temperature of the patient. The SpO2 module includes a clip that attaches to an appendage of the patient and is designed to measure oxygen content within the blood and a pulse rate of the patient. The NIBP module includes an inflatable cuff that attaches to an appendage of the patient and is designed to measure the blood pressure of the patient. In some embodiments, other HCE modules and physiological parameters can also be measured by the monitoring device 102.

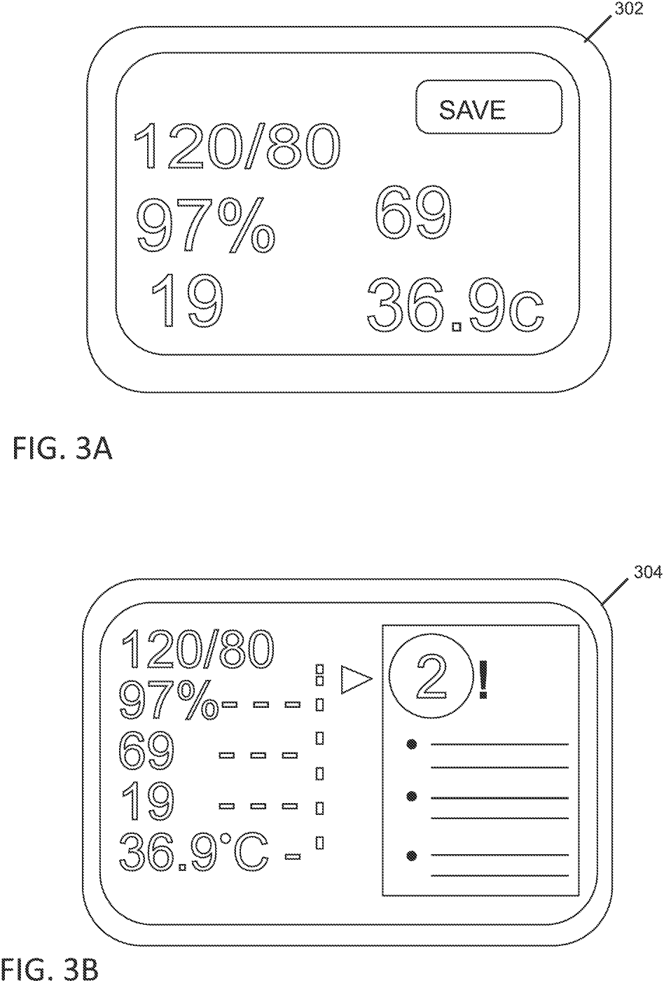

The monitoring device 102 also includes a display to show the measured physiological parameters to the caregiver. For instance, as illustrated in FIG. 3A, the display can provide such information as the blood pressure (systolic and diastolic), oxygen saturation, pulse rate, and temperate.

The monitoring device 102 can operate within one or more workflows. A workflow is a series of one or more tasks that a user of the monitoring device 102 performs. When the monitoring device 102 operates within a workflow, the monitoring device 102 provides functionality suitable for assisting the user in performing the workflow. When the monitoring device 102 operates within different workflows, the monitoring device 102 provides different functionality.

For example, in some embodiments, the monitoring device 102 can operate within a monitoring workflow or a non-monitoring workflow. Example types of non-monitoring workflows include, but are not limited to, a spot check workflow and a triage workflow.

When the monitoring device 102 is operating within the monitoring workflow, the monitoring device 102 obtains a series of measurements of one or more physiological parameters of a single monitored patient over a period of time. In addition, the monitoring device 102 displays a monitoring workflow home screen.

When the monitoring device 102 is operating within a non-monitoring workflow, the monitoring device 102 obtains a measurement of one or more physiological parameters from each patient in a series of patients. In addition, the monitoring device 102 displays a non-monitoring workflow home screen. The non-monitoring workflow home screen contains a representation of the physiological parameter of a given patient in the series of patients.

Examples of the monitoring device 102 include, without limitation, the CONNEX® Vital Signs Monitors (CVSM) and CONNEX® Spot Monitors (CSM) from Welch Allyn, Inc. of Skaneateles Falls, NY. As noted, such devices can communicate with various other devices, such as the server device 112 and/or the Connex® Central Stations (CCS) from Welch Allyn, Inc. using the Connex® system. Additional details regarding the monitoring device 102 is provided in U.S. Pat. No. 9,265,429 filed on Mar. 31, 2010, the entirety of which is hereby incorporated by reference.

The example monitoring device 102 is connected to the server device 112 through the network 108. The network 108 may include any type of wireless network, a wired network, or any communication network known in the art. For example, wireless connections can include cellular network connections and connections made using protocols such as 802.11a, b, and/or g. In other examples, a wireless connection can be accomplished directly between the monitoring device 102 and an external display using one or more wired or wireless protocols, such as Bluetooth, Wi-Fi Direct, radio-frequency identification (RFID), or ZigBee. Other configurations are possible.

The network 108 is an electronic communication network that includes a set of computing devices and links between the computing devices. The computing devices in the network use the links to enable communication among the computing devices in the network. The network 108 can include routers, switches, mobile access points, bridges, hubs, intrusion detection devices, storage devices, standalone server devices, blade server devices, sensors, desktop computers, firewall devices, laptop computers, handheld computers, mobile telephones, and other types of computing devices. In various embodiments, the network 108 includes various types of links. For example, the network 108 can include wired and/or wireless links. Furthermore, in various embodiments, the network 108 is implemented at various scales. For example, the network 108 can be implemented as one or more local area networks (LANs), metropolitan area networks, subnets, wide area networks (such as the Internet), or can be implemented at another scale.

The server device 112 receives data from the monitoring device 102. Such data can include the physiological parameters measured by the monitoring device 102. The data can also include identification information associated with the caregiver and/or the patient. The server device 112 can include artificial intelligence, such as machine learning and/or neural network, that allows the server device 112 to analyze the data from the monitoring device 102 and provide feedback to the caregiver.

In the example shown, the server device 112 is a computing system that allows storage, retrieval, and manipulation of electronic medical records. Example types of computing devices include personal computers, standalone server computers, blade server computers, mainframe computers, handheld computers, smart phones, special purpose computing devices, and other types of devices that process data.

For example, the server device 112 can be implemented as one or more computing devices in the cloud that are networked to provide processing power. The server device 112 can be connected to a database 114 to store and access data from a large number of patients. In some examples, the database 114 is an Electronic Medical Records (EMR) system. The server device 112 can also interface with other computing devices, such as an active directory like the Active Directory directory service from Microsoft Corporation of Redmond, Washington.

The monitoring device 102 can send various types of data to the server device 112 for storage in the database 114 and can receive various types of data from the server device 112. For example, in some embodiments, the monitoring device 102 sends measurements of physiological parameters to the server device 112 for storage in the database 114. In other examples, the monitoring device 102 sends credentials associated with a caregiver to the server device 112 and/or requests historical physiological information from the server device 112.

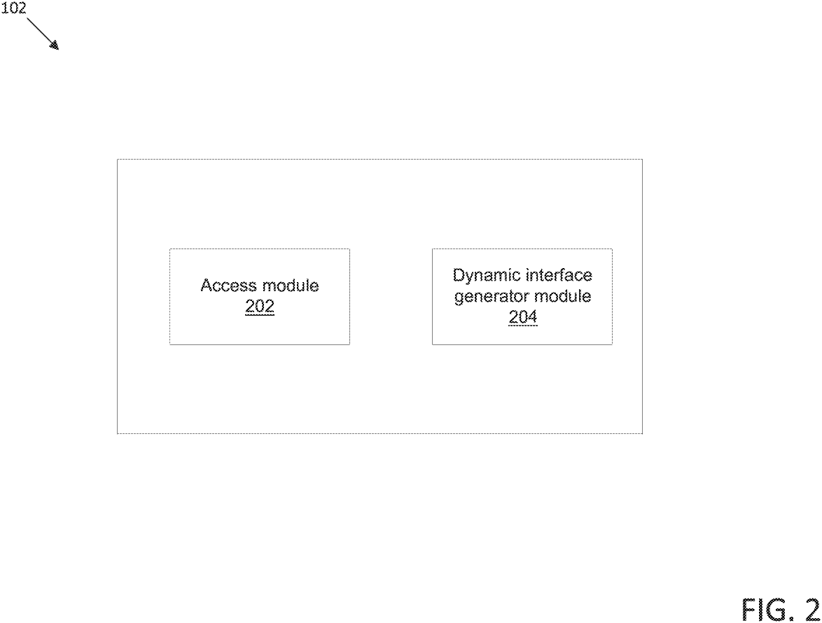

Referring now to FIGS. 2-4, the monitoring device 102 is generally programmed to provide a dynamic interface based on user role. This has clinical benefits of ensuring that caregivers are working in compliance with their scope of practice and facility policy for documentation integrity and may help to ensure patient safety by protecting patients from inappropriate alarm setting changes by underqualified caregivers.

In some examples, the dynamic interface is defined based upon access credentials associated with the caregiver. The access can be segregated into access levels based upon the caregiver’s role, with the dynamic interface being generated based upon the specific access level associated with the caregiver.

More specifically, as shown in FIG. 2, the monitoring device 102 includes an access module 202 and a dynamic interface generator module 204.

The access module 202 generally accesses the credentials associated with the caregiver using the monitoring device 102. The credentials can include, without limitation, data that identifies the caregiver, such as an identification number, password, etc. Such data can be provided by the caregiver by scanning a badge or otherwise inputting the information, such as by using a keyboard or selecting the caregiver’s name from a list.

The access module 202 is programmed to use the identity of the caregiver to define a given level of access. More specifically, in this example, the access module 202 is programmed to send the data associated with the caregiver to the server device 112. As described further below, the server device 112 looks up the caregiver in a directory and identifies an access level for access. This access level is communicated back to the access module 202. The access module 202 thereupon communicates the access level to the dynamic interface generator module 204.

Examples of categories of such roles include, without limitation: (i) nurse assistant; (ii) registered nurse; (iii) nurse manager; and (iv) biomedical device technician. These are just examples, and different roles can be defined by the location (e.g., hospital) and other device manufacturer. Access levels can be added and/or removed as required.

The dynamic interface generator module 204 is programmed to use the access level to define both the level of access given to the caregiver on the monitoring device 102 and the dynamic interface generated for the caregiver on the monitoring device 102. For instance, the dynamic interface generator module 204 defines how the dynamic interface looks on the monitoring device 102, as well as defines what options are provided to the caregiver.

For example, referring to FIGS. 3A and 3B, two dynamic interfaces 302, 304 for the monitoring device 102 are shown.

Generally, the greater the access level, the greater the amount of control and/or information that is displayed for the caregiver. The dynamic interface 302 of the monitoring device 102 shown in FIG. 3A is generated for a lower access level, such as a nurse assistant. The dynamic interface 302 provides basic information, such as the monitored physiological parameters. The dynamic interface 302 simply allows the caregiver to capture and save physiological parameters.

The dynamic interface 304 of the monitoring device 102 shown in FIG. 3B is generated for a higher access level, such as a registered nurse. The dynamic interface 304 provide additional functionality beyond the capturing and saving of physiological parameters. In this example, the dynamic interface 304 also provides early warning score information and alarming associated therewith to the caregiver. The dynamic interface 304 can also provide additional functionality, such as allowing the caregiver to mute or adjust alarms.

Other configurations for different access levels are possible. For instance, a higher access level can be provided for a manager or biomedical device technician. This level can provide interfaces and access to more functionality associated with the monitoring device 102.

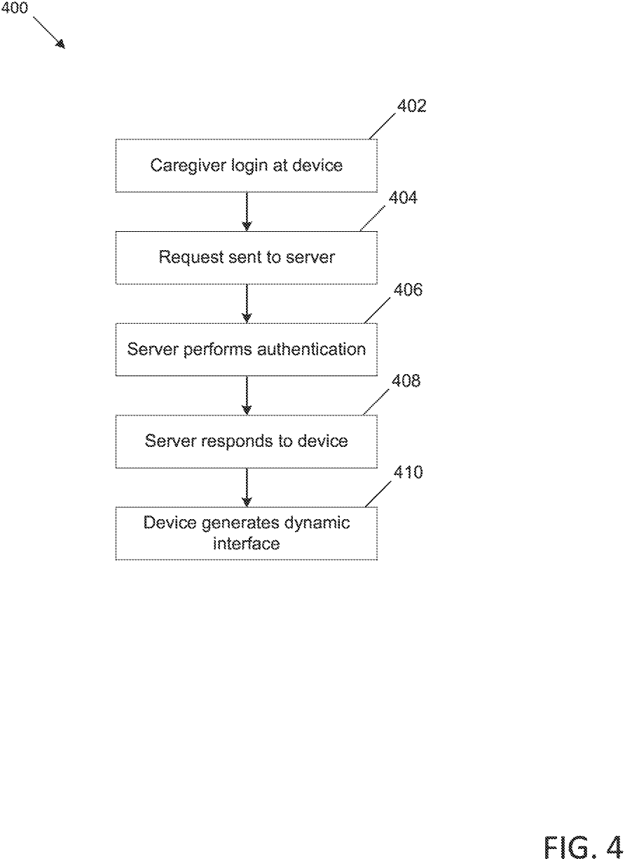

Referring now to FIG. 4, an example method 400 for generating the dynamic interface is provided.

At operation 402, the caregiver provides a login at the monitoring device 102. As noted, the login can include credentials like a caregiver identifier, password, etc. In one example, the caregiver swipes a badge at the monitoring device to provide the credentials. Next, at operation 404 the monitoring device 102 sends the credentials to the server device 112.

The server device 112 thereupon performs authentication at operation 406. This authentication can include the authentication of the credentials and a lookup of the access level associated with the caregiver. In one example, the lookup can include accessing an active directory using a lightweight directory access protocol. The directory responds with the access level associated with the caregiver.

Next, at operation 408, the server device 112 responds to the monitoring device 102 with the result of authentication. This can include the access level for the caregiver. One example of such a response can include the following schema shown in Table 1.

TABLE 1

| Caregiver identifier | Authentication result | Access level |
|---|---|---|
| 111000 | Confirmed | 3 |

In this example, the identifier 111000 for the caregiver who logged into the monitoring device 102 is confirmed, and the access level associated with the caregiver is “3”.

Finally, at operation 410, the monitoring device 102 uses the access level associated with the caregiver to generate the dynamic interface. For instance, assuming the access level “3” is associated with a registered nurse, the monitoring device 102 would generate the dynamic interface 304.

Other configurations are possible. For instance, in an alternative embodiment, the monitoring device 102 can perform a mapping from the access level returned by the server device 112. For instance, access levels 1-12 could be stored in the directory accessed by the server device 112. However, the monitoring device 102 can be programmed with less levels and therefore maps the access level returned by the server device 112 with one of the levels on the monitoring device 102. For instances, server access levels 1-3 could be mapped to level 1 on the monitoring device 102, while server access levels 4-6 are mapped to level 2 on the monitoring device 102, and so on.

In yet other embodiments, the access levels can be stored locally on the monitoring device 102 so that the monitoring device 102 does not need to communicate with the server device 112 to generate the dynamic interface. For instance, the monitoring device 102 can store the credentials and associated access levels for caregivers locally, and the monitoring device 102 can access the access levels when creating the dynamic interface upon login by the caregiver. In such an example, the local directory of access levels can be periodically updated through requests to the server device 112. Many other configurations are possible.

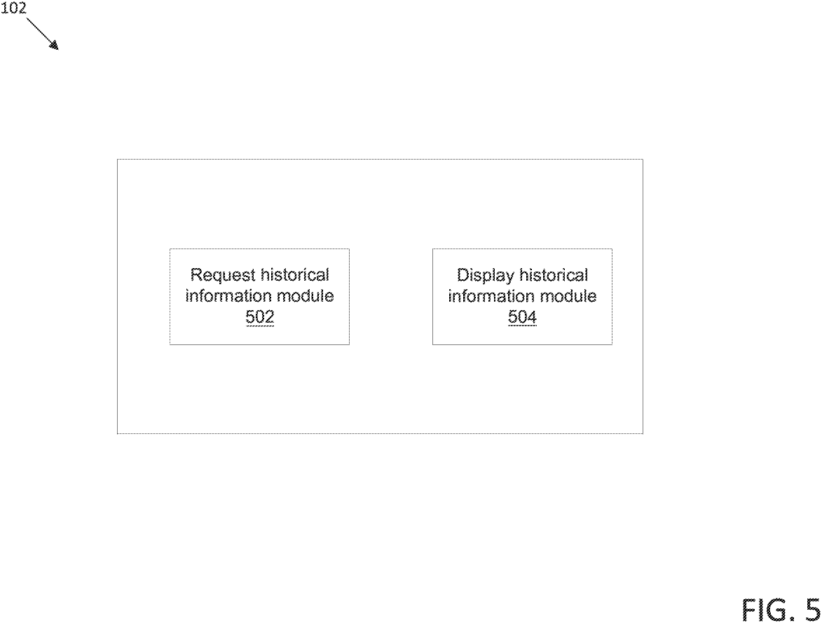

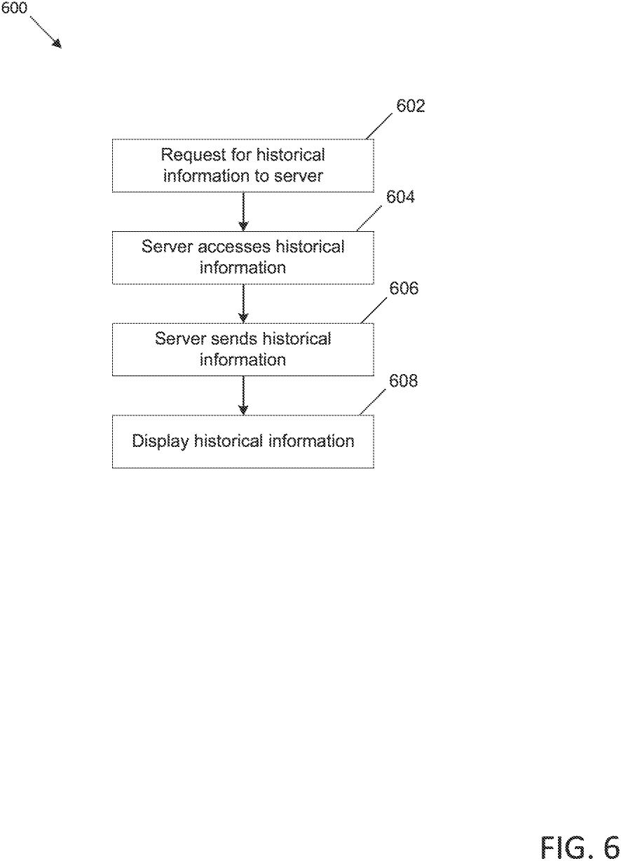

Referring now to FIGS. 5-6, the monitoring device 102 is generally programmed to access historical physiological information associated with the patient to provide additional information to the caregiver. For instance, the monitoring device 102 can access information like historical physiological information from the server device 112 and display this historical physiological information on the monitoring device 102. This allows for information to be accessed that was captured at earlier points in time and/or by other devices. This can generally allow the caregiver to more easily identify trends associated with the patient’s care.

More specifically, as shown in FIG. 5, the monitoring device 102 includes a request historical physiological information module 502 and a display historical physiological information module 504.

The request historical physiological information module 502 is programmed to request historical physiological information associated with the patient. The request can be initiated automatically or manually. For instance, when the caregiver accesses certain information about the patient on the monitoring device 102 (e.g., pulse rate information), the request historical physiological information module 502 is programmed to request historical physiological information (e.g., historical pulse rate from the EMR system). Alternatively, the caregiver can explicitly request historical physiological information, such as by requesting the monitoring device 102 to display pulse rate from a certain day in the past. Many configurations are possible.

Whether the request is automated or manual, the request historical physiological information module 502 is programmed to communicate with the server device 112 to obtain the historical physiological information. For instance, the following schema shown in Table 2 is one illustration of an example request for historical physiological information regarding the patient’s pulse rate.

TABLE 2

| Patient identifier | Historical physiological information requested |
|---|---|
| 1000 | Pulse rate |

In this example, the monitoring device 102 requests historical physiological information relating to pulse rate for the patient (identified with number “1000”) from the server device 112.

The server device 112 can, in turn, obtain the requested historical physiological information based upon the identification of the patient. In one example, the server device 112 accesses the resulted historical pulse rate information from the database 114. In other examples, the server device 112 can access an EMR system to obtain the requested historical physiological information. The server device 112, in turn, returns the historical physiological information to the monitoring device 102.

The display historical physiological information module 504 is programmed to display the historical physiological information. In some examples, the historical physiological information is displayed in a table accessed by the caregiver, such as by selecting the current pulse rate (“69”) on the dynamic interface 304. One example of such is shown in Table 3 provided below.

TABLE 3

| Date/Time | Pulse rate |
|---|---|
| Nov. 3, 2021 - 11:10am | 69 |
| Nov. 3, 2021 - 03:05pm | 68 |
| Nov. 3, 2021 - 07:12pm | 66 |
| Nov. 4, 2021 - 12:25am | 67 |
| Nov. 4, 2021 - 6:03am | 71 |

In the example table, each entry includes date, time of day, and the pulse rate. Additional information associate with the historical physiological information, such as how the information was calculated, can also be provided. In other examples, the historical pulse rate information can be noted to be a daily average.

Many configurations are possible. For instance, in other embodiments, historical graphs and other ways to display trending information is created using the historical physiological information and displayed for the caregiver on the monitoring device 102.

In some examples, the raw historical data is returned from the server device 112 to the monitoring device 102. The raw historical data can include the actual measurements that were recorded. The monitoring device 102, in turn, interprets and displays the historical physiological information. In other examples, the historical physiological information is summarized before being returned to the monitoring device 102 for display.

The amount of historical physiological information that is returned by the server device 112 is configurable. For instance, the server device 112 can be programmed to return the last 12 hours, 24 hours, or 48 hours of historical physiological information. In yet other embodiments, the amount is determined based upon the size of the data, such as how much data is included in the historical physiological information. In yet other embodiments, the amount is dependent on the type of information requested. For some types of information, only a week is provided (e.g., blood pressure), while other types of information may provide for a month (e.g., pulse rate). Many configurations are possible.

The display historical physiological information module 504 can also be programmed to provide enhanced information to the caregiver. For instance, the display historical physiological information module 504 can be programmed to suggest alarm limits based upon the historical physiological information. When the caregiver sets pulse rate alarm limits, the upper and lower thresholds can be suggested by the monitoring device 102 based upon the historical physiological information. For instance, if the patient’s pulse rate is historically fast, the display historical physiological information module 504 can suggest upper thresholds that are higher based upon that historical physiological information.

Referring now to FIG. 6, an example method 600 for accessing historical physiological information is provided.

At operation 602, the request for historical physiological information is sent by the monitoring device 102 to the server device 112. Next, at operation 604, the server device 112 accesses the requested historical physiological information from, for example, a database or other EMR system.

At operation 606, the server device 112 sends the historical physiological information back the monitoring device 102. Finally, at operation 608, the monitoring device 102 displays the historical physiological information. As noted, this can be done in raw form, summary form, trends, and/or other ways.

In some examples, the type and amount of historical physiological information that can be accessed is based upon the access level for the caregiver. For instance, the lower access level may not even be allowed to request or access historical physiological information. However, a higher access level may be allowed to request some or all the historical physiological information that is available. The access can be configurable, as desired.

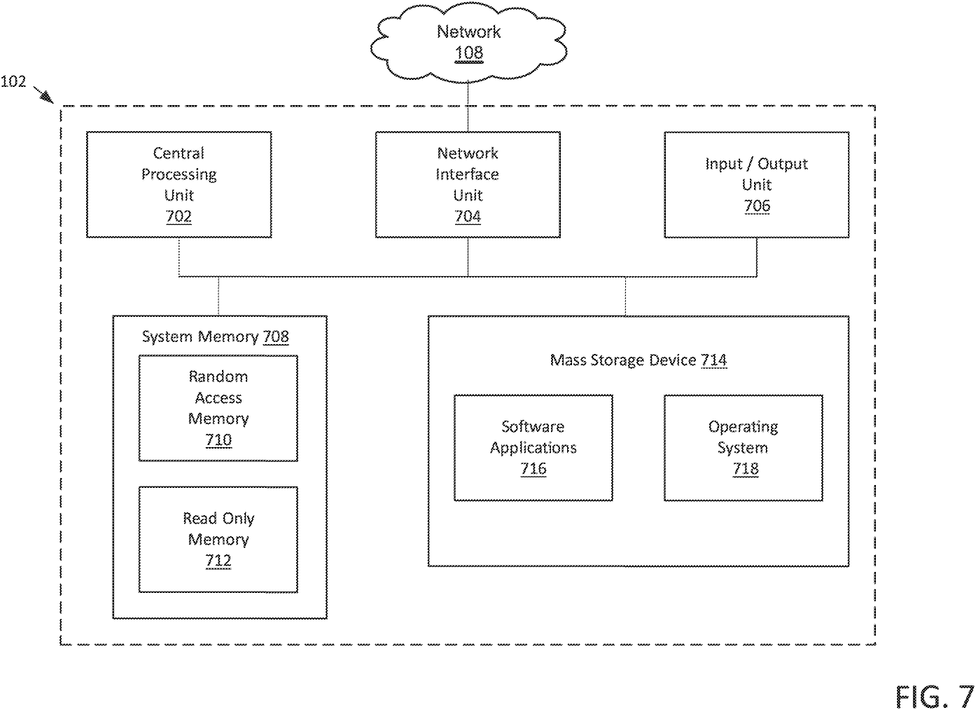

Referring now to FIG. 7, example components of the monitoring device 102 are provided. The server device 112 can include similar components.

The monitoring device 102 may have one or more input devices and one or more output devices configured to communicate with an input / output unit 706. The input devices can be a camera, a keyboard, a mouse, a pen, a sound or voice input device, a touch or swipe input device, a barcode scanner, or a radio-frequency identification scanner, etc. Output devices such as a display, speakers, a printer, an alarm light bar, etc. may also be included. The aforementioned devices are examples and others may be used. The monitoring device 102 may include a network interface unit 704 allowing communications with other computing devices. Examples include, but are not limited to, RF transmitter, receiver, and/or transceiver circuitry; universal serial bus (USB), parallel, and/or serial ports.

The monitoring device 102 includes system memory 708 and a mass storage device 714, both of which can include computer readable media. The term computer readable media as used herein may include non-transitory computer readable media. The system memory 708 may include volatile and nonvolatile, removable and non-removable media implemented in any method or technology for storage of information, such as computer readable instructions, data structures, or program modules. For example, the system memory 708 can include Random Access Memory (RAM) 710 and Read Only Memory (ROM) 712.

The mass storage device 714 may also include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information, and which can be accessed by the monitoring device 102. Any such computer storage media may be part of the monitoring device 102.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

The monitoring device 102 also includes a central processing unit 702 (also referred to as a processor) to execute an operating system 718 and/or one or more software applications 716 stored on the mass storage device 714. For example, the central processing unit 702 can execute the software applications 716 to provide one or more workflows that allow the caregiver to accomplish the functionality described herein.

The block diagrams depicted herein are just examples. There may be many variations to these diagrams described therein without departing from the spirit of the disclosure. For instance, components may be added, deleted or modified.

While embodiments have been described, it will be understood that those skilled in the art, both now and in the future, may make various improvements and enhancements can be made. The description and illustration of one or more embodiments provided in this application are not intended to limit or restrict the scope of the invention as claimed in any way. The embodiments, examples, and details provided in this application are considered sufficient to convey possession and enable others to make and use the best mode of claimed invention.

The claimed inventions should not be construed as being limited to any embodiment, example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate embodiments falling within the spirit of the broader aspects of the claimed inventions and the general inventive concepts embodied herein.

What is claimed is:

1. A monitoring device, comprising:
at least one processor; and
at least one system memory encoding instructions which, when executed by the at least one processor, cause the monitoring device to:
receive an identifier for a caregiver;
determine an access level for the monitoring device based upon the identifier of the caregiver;
generate a dynamic interface, the dynamic interface being configured based upon the access level of the caregiver;
access historical physiological information based upon the access level; and
suggest one or more alarm limits based upon the historical physiological information.

2. The monitoring device of claim 1, wherein the identifier includes one or more caregiver roles.

3. The monitoring device of claim 1, wherein an increase in the access level provides an increased amount of information that is displayed by the monitoring device.

4. The monitoring device of claim 1, wherein an increase in the access level provides an increased amount of control over the monitoring device.

5. The monitoring device of claim 1, comprising further instructions which, when executed by the at least one processor, cause the monitoring device to:
restrict access to control over alarm settings based upon the access level.

6. The monitoring device of claim 1, comprising further instructions which, when executed by the at least one processor, cause the monitoring device to:
send the identifier to a server device; and
receive the access level from the server device.

7. The monitoring device of claim 1, comprising further instructions which, when executed by the at least one processor, cause the monitoring device to:
capture physiological parameters of a patient.

8. The monitoring device of claim 1, comprising further instructions which, when executed by the at least one processor, cause the monitoring device to:
suggest upper and lower alarm limits based upon the historical physiological information.

9. A monitoring device, comprising:
at least one processor; and
at least one system memory encoding instructions which, when executed by the at least one processor, cause the monitoring device to:
request historical physiological information from a second device;
receive the historical physiological information;
display the historical physiological information on the monitoring device; and
suggest one or more alarm limits based upon the historical physiological information.

10. The monitoring device of claim 9, wherein the historical physiological information includes one or more vital signs.

11. The monitoring device of claim 9, wherein the second device is a server device.

12. The monitoring device of claim 9, wherein the historical physiological information is displayed in a tabular format.

13. The monitoring device of claim 9, wherein the historical physiological information is displayed in a graphical format.

14. The monitoring device of claim 9, wherein the historical physiological information includes raw physiological parameters.

15. A method for monitoring a patient, the method comprising:
receiving an identifier for a caregiver;
determining an access level for a monitoring device based upon the identifier of the caregiver;
receiving historical physiological information for the patient;
generating a dynamic interface, the dynamic interface being configured based upon the access level of the caregiver;
displaying the historical physiological information, wherein display of the historical physiological information is limited by the access level of the caregiver; and
suggesting one or more alarm limits based upon the historical physiological information.

16. The method of claim 15, further comprising:
sending the identifier to a server device; and
receiving the access level from the server device.

17. The method of claim 15, further comprising capturing physiological parameters of the patient.

18. The method of claim 15, further comprising displaying the historical physiological information in a tabular format.

19. The method of claim 15, further comprising displaying the historical physiological information in a graphical format.

20. The method of claim 15, wherein the historical physiological information includes raw physiological parameters.
